(19) United States
(12) Patent Application Publication
(10) Pub. No.: US 2026/0186005 A1
(43) Pub. Date: Jul. 2, 2026

(54) SENSOR FOR DETECTING A PLURALITY OF ANALYTES

(71) Applicant: Roche Diagnostics Operations, Inc., Indianapolis, IN (US)

(72) Inventors: Ildiko AMANN-ZALAN, Weinheim-Hohensachsen (DE); Oscar GUTIÉRREZ-SANZ, Heidelberg (DE); Eloisa LOPEZ-CALLE, Ludwigshafen (DE); Christopher PROBST, Esslingen am Neckar (DE); Robin WAGNER, Ludwigshafen (DE)

(21) Appl. No.: 19/546,052

(22) Filed: Feb. 20, 2026

Related U.S. Application Data
(63) Continuation of application No. PCT/EP2024/073193, filed on Aug. 19, 2024.

(30) Foreign Application Priority Data
Aug. 22, 2023 (EP) 23192699.9

(51) Int. Cl. G01N 33/70; G01N 27/30; G01N 27/327; G01N 33/543; G01N 33/84

(52) U.S. Cl. CPC G01N 33/70; G01N 27/301; G01N 27/3275; G01N 33/5438; G01N 33/84

(57) ABSTRACT

There is disclosed a method and sensor for detecting a plurality of analytes in bodily fluid, the sensor comprising electrodes including a first working electrode, a second working electrode, a third working electrode, a counter electrode, and a reference electrode; wherein the first working electrode is configured to detect a first signal indicative of a first analyte concentration using potentiometry; wherein the second working electrode is configured to detect a second signal indicative of a second analyte concentration using amperometry; wherein the third working electrode is configured to detect a third signal indicative of a third analyte concentration using amperometry; wherein the first analyte, the second analyte, and third analyte are different analytes; wherein the first signal, the second signal, and the third signal are detected using the same reference electrode; wherein at least part of the detection of the first signal occurs simultaneously with the detection of the second signal; wherein the second signal and third signal are detected using the same counter electrode; wherein at least part of the detection of the second signal occurs simultaneously with the detection of the third signal; and wherein at least one of the second working electrode and third working electrode is continuously polarized by application of a polarization voltage during detection of the first signal, second signal and third signal.

### CLAIM OF PRIORITY

This application is a continuation of PCT/EP2024/073193 (filed on Aug. 19, 2024), which claims priority to and benefit of European Patent Application no. 23192699.9 (filed on Aug. 22, 2023). The contents of these applications are incorporated herein by reference in their entirety.

### TECHNICAL FIELD

Certain examples of the present disclosure relate to a sensor for detecting a plurality of analytes in bodily fluid, and a method of detecting a plurality of analytes in bodily fluid using the sensor.

### BACKGROUND

Wearable sensing technologies have been of interest in recent years due to the huge amount of possibilities they could bring to healthcare. However, despite the large number of technologies in the market, not many are being used in professional health care.

One of the most revolutionary technologies in this field is continuous glucose monitoring. This technology helped millions of diabetic patients to easily monitor their glucose by wearing an minimally invasive patch able to measure their glucose levels in subcutaneous interstitial fluid.

This technology is usually based on a redox reaction that can be monitored by means of electrochemistry. However, not all chemical biomarkers can be detected by following a redox reaction as in the case of glucose.

Furthermore, given the complexity of human biology, simply monitoring one parameter over time is not always enough.

Therefore there is a need to have the possibility of monitoring different chemical parameters so that this technology could be designed to be used in cases in which just one parameter does not provide enough information to trigger accurate medical decisions.

D. Ma, S. S. Ghoreishizadeh and P. Georgiou, “Concurrent Potentiometric and Amperometric Sensing With Shared Reference Electrodes,” in IEEE Sensors Journal, vol. 21, no. 5, pp. 5720-5727, 1 Mar. 2021, doi: 10.1109/JSEN.2020.3039567 investigates concurrent potentiometry and amperometry through the means of a shared reference electrode. pH and hydrogen peroxide are selected as target analytes for potentiometric and amperometric measurements respectively.

The above information is presented as background information only to assist with an understanding of the present disclosure. No determination has been made, and no assertion is made, as to whether any of the above might be applicable as prior art with regard to the present invention.

### SUMMARY

It is an aim of certain examples of the present disclosure to address, solve and/or mitigate, at least partly, at least one of the problems and/or disadvantages associated with the related art, for example at least one of the problems and/or disadvantages described herein.

It is an aim of certain examples of the present disclosure to provide a sensor and method for detecting a plurality of analytes in bodily fluid. A particular aim of certain examples is to provide a compact sensor capable of detecting a plurality of analytes using a combination of potentiometric and amperometric techniques.

A sensor for detecting a plurality of analytes in bodily fluid comprises electrodes including a first working electrode, a second working electrode, a third working electrode, a counter electrode, and a reference electrode. The first working electrode is configured to detect a first signal indicative of a first analyte concentration using potentiometry. The second working electrode is configured to detect a second signal indicative of a second analyte concentration using amperometry. The third working electrode is configured to detect a third signal indicative of a third analyte concentration using amperometry. The first, second, and third analytes are different analytes. Advantageously, this allows multiple different analyte concentrations to be detected using a single sensor.

The first signal, the second signal, and the third signal are detected using the same reference electrode. By sharing electrodes for detecting multiple analytes, the size, cost, and manufacturing complexity of the sensor may be reduced.

At least part of the detection of the second signal occurs simultaneously with the detection of the first signal.

Advantageously, this allows simultaneous performance of potentiometric measurement at the first working electrode and amperometric measurement at the second working electrode using the same reference electrode.

Consequently, a compact sensor for simultaneously detecting a plurality of analytes may be provided.

The second signal and third signal are detected using the same counter electrode. By sharing electrodes for detecting multiple analytes, the size, cost, and manufacturing complexity of the sensor may be reduced.

At least part of the detection of the second signal occurs simultaneously with the detection of the third signal.

Advantageously, this allows simultaneous performance of amperometric measurements at the second working electrode and the third working electrode using the same counter electrode and the same reference electrode.

Consequently, a compact sensor for simultaneously detecting a plurality of analytes may be provided.

At least one of the second working electrode and third working electrode is continuously polarized by application of a polarization voltage during detection of the first signal, second signal and third signal.

Advantageously, this avoids the need to re-polarize the second working electrode and/or third working electrode before each measurement.

Consequently, a measurement frequency of a compact sensor for simultaneously detecting a plurality of analytes may be improved.

Optionally, at least part of the detection of the third signal may occur simultaneously with detection of the first signal and the second signal.

Advantageously, this allows simultaneous performance of potentiometric measurement at the first working electrode and amperometric measurements at the second working electrode and third working electrode using a single shared reference electrode and a single shared counter electrode.

Consequently, a compact sensor for simultaneously detecting a plurality of analytes may be provided. For example, a sensor for simultaneously detecting three different analytes using only five electrodes may be provided by sharing a single reference electrode and a single counter electrode between three working electrodes.

Optionally, the first analyte is an ion.

Optionally, the first analyte is one of potassium, sodium, magnesium, urea, or calcium.

Optionally, the second analyte is a metabolite.

Optionally, the second analyte is one of creatinine, creatine, NT-proBNP, BNP, glucose, lactate, ketone, alcohol, or oxygen.

Optionally, the third analyte is a metabolite.

Optionally, the third analyte is one of creatinine, creatine, NT-proBNP, BNP, glucose, lactate, ketone, alcohol, or oxygen.

Optionally, the third signal is used to correct the second signal.

Advantageously, this allows for more accurate determination of the second analyte concentration.

Optionally, the first analyte is potassium, the second analyte is creatinine, and the third analyte is creatine.

Potassium and creatinine are biomarkers relevant to monitoring kidney function and detecting renal failure. Advantageously, the use of the third working electrode to detect creatine concentration allows the creatinine concentration detected by the second working electrode to be corrected to remove any influence from endogenous creatine on the second signal. Consequently, more accurate monitoring of kidney function may be performed.

Optionally, at least one of the first, second, or third analyte concentrations is indicative of a health condition, and wherein at least one other of the first, second, or third analyte concentrations is indicative of a side effect of medication for treating the health condition.

Advantageously, by simultaneously detecting a signal indicative of the intended effect of a treatment and a signal indicative of the side effects of treatment, the treatment may be used more effectively.

Optionally, the sensor is a needle-type sensor comprising an electrode area on which the first working electrode, second working electrode, third working electrode, counter electrode, and reference electrode are arranged; wherein the electrode area comprises a first surface facing a first direction on which at least one of the first working electrode, second working electrode, third working electrode, counter electrode, and reference electrode are arranged; wherein the electrode area comprises a second surface facing a second direction, opposite to the first direction, on which is arranged at least one of the first working electrode, second working electrode, third working electrode, counter electrode, and reference electrode that is not arranged on the first surface.

Advantageously, the use of both sensor surfaces reduces the width of the electrode area to facilitate implantation of the needle-type sensor into tissue. Consequently, a compact sensor for simultaneously detecting a plurality of analytes in tissue may be provided.

Optionally, the first working electrode is arranged on the second surface and the second working electrode, third working electrode, and counter electrode are arranged on the first surface.

Advantageously, by arranging the first working electrode on a different surface to the second working electrode, third working electrode, and counter electrode, interference between the potentiometric and amperometric signals may be reduced.

Consequently, a sensitivity of a compact sensor for simultaneously detecting a plurality of analytes may be improved.

Optionally, at least one of the electrodes arranged on the first surface extends across the full width of the first surface and/or at least one of the electrodes arranged on the second surface extends across the full width of the second surface.

Optionally, each of the electrodes arranged on the first surface extends across the full width of the first surface and each of the electrodes arranged on the second surface extends across the full width of the second surface.

Advantageously, the use of the full width of the electrode area surface to maximise the size of some or all of the electrodes improves the signal strength.

Consequently, a sensitivity of a compact sensor for simultaneously detecting a plurality of analytes may be improved.

In a method of detecting a plurality of analytes in bodily fluid using a sensor comprising a first working electrode, a second working electrode, a third working electrode, a counter electrode, and a reference electrode, the following steps are performed. In a first step, a first signal indicative of a first analyte concentration is detected using potentiometry at the first working electrode. In a second step, a second signal indicative of a second analyte concentration is detected using amperometry at the second working electrode. In a third step, a third signal indicative of a third analyte concentration is detected using amperometry at the third working electrode. The first analyte, second analyte, and third analyte are different analytes. Advantageously, this allows multiple analyte concentrations to be detected using a single sensor.

The first signal, second signal, and third signal are detected using the same reference electrode. By sharing electrodes for detecting multiple analytes, the size, cost, and manufacturing complexity of the sensor may be reduced.

At least part of the detection of the second signal occurs simultaneously with the detection of the first signal.

Advantageously, this allows simultaneous performance of potentiometric measurement at the first working electrode and amperometric measurement at the second working electrode using the same reference electrode.

Consequently, a method of using a compact sensor for simultaneously detecting a plurality of analytes may be provided.

The second signal and third signal are detected using the same counter electrode. By sharing electrodes for detecting multiple analytes, the size, cost, and manufacturing complexity of the sensor may be reduced.

At least part of the detection of the second signal occurs simultaneously with the detection of the third signal.

Advantageously, this allows simultaneous performance of amperometric measurements at the second working electrode and the third working electrode using the same counter electrode and the same reference electrode.

Consequently, a method of using a compact sensor for simultaneously detecting a plurality of analytes may be provided.

The method further comprises continuously polarizing at least one of the second working electrode and third working electrode by application of a polarization voltage during detection of the first signal, second signal and third signal.

Advantageously, this avoids the need to re-polarize the second working electrode and/or third working electrode before each measurement.

Consequently, a method of using a compact sensor for simultaneously detecting a plurality of analytes with improved measurement frequency may be provided.

Optionally, at least part of the detection of the third signal may occur simultaneously with detection of the first signal and the second signal.

Advantageously, this allows simultaneous performance of potentiometric measurement at the first working electrode and amperometric measurements at the second working electrode and third working electrode using a single shared reference electrode and a single shared counter electrode.

Consequently, a method of using a compact sensor for simultaneously detecting a plurality of analytes may be provided. For example, a method of using a sensor for simultaneously detecting three different analytes using only five electrodes may be provided by sharing a single reference electrode and a single counter electrode between three working electrodes.

Optionally, the method further comprises monitoring the first analyte concentration, based on the first signal, monitoring the second analyte concentration, based on the second signal, and monitoring the third analyte concentration, based on the third signal.

Advantageously, this allows for monitoring of multiple analyte concentrations with a single sensor.

Optionally, the method further comprises monitoring the first analyte concentration, based on the first signal; monitoring the second analyte concentration, based on the second signal and the third signal, by correcting the second signal using the third signal.

Advantageously, this allows for more accurate monitoring of the second analyte concentration.

Optionally, the first analyte is potassium, the second analyte is creatinine, and the third analyte is creatine.

Potassium and creatinine are biomarkers relevant to monitoring kidney function and detecting renal failure. Advantageously, the use of the third working electrode to detect creatine concentration allows the creatinine concentration detected by the second working electrode to be corrected to remove any influence from endogenous creatine on the second signal. Consequently, more accurate monitoring of kidney failure may be performed.

Optionally, at least one of the first, second, or third analyte concentrations is indicative of a health condition, and wherein at least one other of the first, second, or third analyte concentrations is indicative of a side effect of medication for treating the health condition.

Advantageously, by simultaneously detecting a signal indicative of the intended effect of a treatment and a signal indicative of the side effects of treatment, the treatment may be used more effectively.

Optionally, the sensor is a needle-type sensor comprising an electrode area on which the first working electrode, second working electrode, third working electrode, counter electrode, and reference electrode are arranged; wherein the electrode area comprises a first surface facing a first direction on which at least one of the first working electrode, second working electrode, third working electrode, counter electrode, and reference electrode are arranged; wherein the electrode area comprises a second surface facing a second direction, opposite to the first direction, on which is arranged at least one of the first working electrode, second working electrode, third working electrode, counter electrode, and reference electrode that is not arranged on the first surface.

Advantageously, the use of both sensor surfaces reduces the width of the electrode area to facilitate implantation of the needle-type sensor into tissue. Consequently, a compact sensor for simultaneously detecting a plurality of analytes in tissue may be provided.

Optionally, the first working electrode is arranged on the second surface and the second working electrode, third working electrode, and counter electrode are arranged on the first surface.

Advantageously, by arranging the first working electrode on a different surface to the second working electrode, third working electrode, and counter electrode, interference between the potentiometric and amperometric signals may be reduced.

Consequently, a sensitivity of a compact sensor for simultaneously detecting a plurality of analytes may be improved.

Optionally, at least one of the electrodes arranged on the first surface extends across the full width of the first surface and/or at least one of the electrodes arranged on the second surface extends across the full width of the second surface.

Optionally, each of the electrodes arranged on the first surface extends across the full width of the first surface and each of the electrodes arranged on the second surface extends across the full width of the second surface

Advantageously, the use of the full width of the electrode area surface to maximise the size of some or all of the electrodes improves the signal strength.

Consequently, a sensitivity of a compact sensor for simultaneously detecting a plurality of analytes may be improved.

Embodiments or examples disclosed in the description and/or figures falling outside the scope of the claims are to be understood as examples useful for understanding the present invention.

Other aspects, advantages, and salient features of the invention will become apparent to those skilled in the art from the following detailed description taken in conjunction with the accompanying drawings.

### BRIEF DESCRIPTION OF THE DRAWINGS

FIG. 1 illustrates an example sensor for detecting a plurality of analytes in bodily fluid;

FIG. 2A illustrates an example electrode arrangement in an electrode area of the sensor;

FIG. 2B illustrates an example electrode arrangement of the sensor;

FIG. 3 illustrates an example method of detecting a plurality of analytes in bodily fluid;

FIG. 4 illustrates an example method of detecting a plurality of analytes in bodily fluid; and

FIGS. 5A and 5B show experimental results demonstrating use of the sensor.

### DETAILED DESCRIPTION

The following description of examples of the present disclosure, with reference to the accompanying drawings, is provided to assist in a comprehensive understanding of the present invention, as defined by the claims. The description includes various specific details to assist in that understanding but these are to be regarded as merely exemplary. Accordingly, those of ordinary skill in the art will recognize that various changes and modifications of the examples described herein can be made.

The same or similar components may be designated by the same or similar reference numerals, although they may be illustrated in different drawings.

Detailed descriptions of techniques, structures, constructions, functions or processes known in the art may be omitted for clarity and conciseness, and to avoid obscuring the subject matter of the present disclosure.

The terms and words used herein are not limited to the bibliographical or standard meanings, but, are merely used to enable a clear and consistent understanding of the examples disclosed herein.

Throughout the description and claims, the words “comprise”, “contain” and “include”, and variations thereof, for example “comprising”, “containing” and “including”, means “including but not limited to”, and is not intended to (and does not) exclude other features, elements, components, integers, steps, processes, functions, characteristics, and the like.

Throughout the description and claims, the singular form, for example “a”, “an” and “the”, encompasses the plural unless the context otherwise requires. For example, reference to “an object” includes reference to one or more of such objects.

Throughout the description and claims, language in the general form of “X for Y” (where Y is some action, process, function, activity or step and X is some means for carrying out that action, process, function, activity or step) encompasses means X adapted, configured or arranged specifically, but not necessarily exclusively, to do Y.

Features, elements, components, integers, steps, processes, functions, characteristics, and the like, described in conjunction with a particular aspect, embodiment, example or claim are to be understood to be applicable to any other aspect, embodiment, example or claim disclosed herein unless incompatible therewith.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. It is to be understood that this invention is not limited to the particular methodology, protocols, and reagents described, as these may vary, depending upon the context they are used by those of skill in the art.

While the invention has been shown and described with reference to certain examples, it will be understood by those skilled in the art that various changes in form and detail may be made therein without departing from the scope of the invention, as defined by the appended claims.

Certain examples of the present disclosure provide one or more techniques as disclosed in the appended annex to the description. The skilled person will appreciate that any of these techniques may be applied in combination with any of the techniques described above and illustrated in the Figures.

Throughout the description and claims, the term “analyte” is used to denote a substance of interest, which the sensors described herein are designed to detect. Detecting an analyte may comprise detecting a signal indicative of the presence or concentration of the analyte. An analyte may be any substance of interest that may be present in a sample, for example a molecule, an ion, a biomarker, a metabolite, a peptide, a protein, an antigen, or any other substance intended to be detected by the sensor. The presence or concentration of an analyte in bodily fluids may be indicative of physiological and/or medical states and conditions in the body.

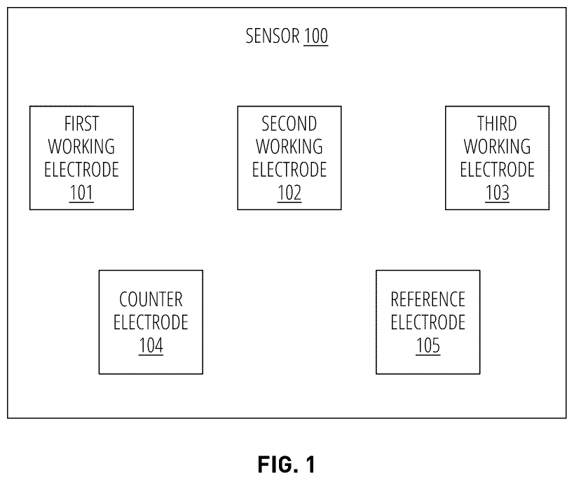

FIG. 1 illustrates an example sensor 100 for detecting a plurality of analytes in bodily fluid. It will be appreciated that the sensor 100 illustrated in FIG. 1 is merely an example, and that the sensor and electrodes are not limited to the particular positions, shapes, sizes, or other characteristics shown in FIG. 1.

The sensor 100 may comprise electrodes. The electrodes may include a first working electrode 101, a second working electrode 102, a third working electrode 103, a counter electrode 104, and a reference electrode 105.

The first working electrode 101 may be configured to detect a first signal indicative of a first analyte concentration using potentiometry. That is, the sensor 100 may be configured to perform a potentiometric measurement using the first working electrode 101 in order to detect a first signal (e.g. a potential), wherein the first signal may represent or be used to calculate a concentration of a first analyte.

The second working electrode 102 may be configured to detect a second signal indicative of a second analyte concentration using amperometry. That is, the sensor 100 may be configured to perform an amperometric measurement using the second working electrode 102 in order to detect a second signal (e.g. a current), wherein the second signal may represent or be used to calculate a concentration of a second analyte.

The third working electrode 103 may be configured to detect a third signal indicative of a third analyte concentration using amperometry. That is, the sensor 100 may be configured to perform an amperometric measurement using the third working electrode 103 in order to detect a third signal (e.g. a current), wherein the third signal may represent or be used to calculate a concentration of a third analyte.

The first analyte may be different to the second analyte, and the third analyte may be different to the first analyte and the second analyte. That is, the sensor 100 may be configured to detect an analyte using a potentiometric measurement, and to detect two further different analytes using amperometric measurements.

The first signal, the second signal, and the third signal may be detected using the same reference electrode 105, and the second signal and the third signal may be detected using the same reference electrode 105 (i.e. the same reference electrode 105 as the first signal) and the same counter electrode 104 (i.e. the same counter electrode 104 as each other). For example, the sensor 100 may be configured to perform a potentiometric measurement using the first working electrode 101 with the reference electrode 105, to perform an amperometric measurement using the second working electrode 102 with the counter electrode 104 and the reference electrode 105, and to perform an amperometric measurement using the third working electrode 103 with the counter electrode 104 and the reference electrode 105.

For example, the potentiometric measurement of the first signal may be an open circuit potential measurement of the first working electrode 101 measured against the reference electrode 105, the amperometric measurement of the second signal may be a measurement of the current flowing between the second working electrode 102 and the counter electrode 104 when a constant potential is applied as measured against the same reference electrode 105, and the amperometric measurement of the third signal may be a measurement of the current flowing between the third working electrode 103 and the same counter electrode 104 when a constant potential is applied as measured against the same reference electrode 105. However, it will be appreciated that this combination is merely an example, and that other combinations of potentiometric and amperometric measurements that share counter electrodes and/or reference electrodes are possible.

Advantages of sharing a counter electrode 104 and/or a reference electrode 105 between different techniques may include reductions in the size, cost, and manufacturing complexity of the sensor 100. For example, an amperometric sensor comprising a first working electrode, counter electrode and reference electrode can be modified to detect two additional analytes (one with amperometry and one with potentiometry) by adding just two further working electrodes and sharing the existing counter electrode and reference electrode.

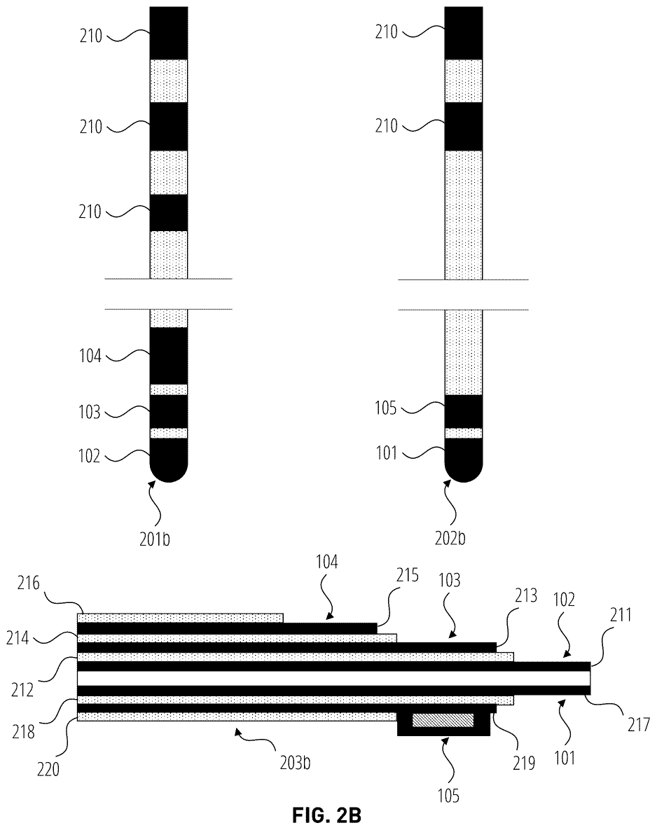

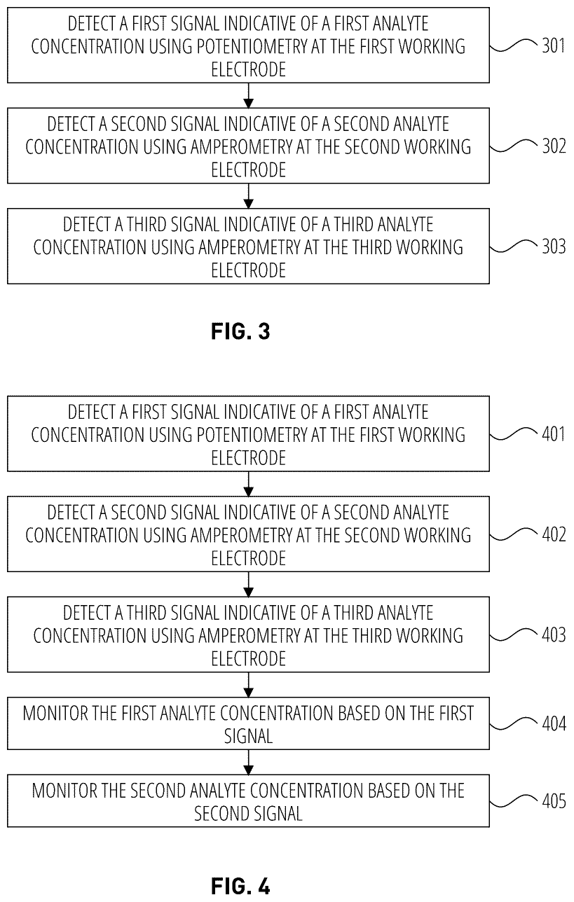

The sensor 100 may further comprise, or may be configured to be connectable to, a control system. The control system may comprise hardware and/or software for controlling the measurements using the sensor 100. The control system may be configured to apply voltages and currents to the electrodes of the sensor 100 to perform the measurements of the first, second, and third signals. The control system may be configured to detect, receive, and/or process the first, second, and third signals. For example, the control system may comprise a potentiostat or other control and measuring device. The control system may further comprise, or be configured to be connectable to, a processor or computer configured to control the potentiostat or other control and measuring device. For example, the sensor 100 may be connected to a control and measuring device (e.g. a potentiostat) under the control of a computing device, and a user may configure and perform the measurements using the computing device and receive the first, second, and third signals (or data derived therefrom) at the computing device. For example, the sensor 100 may comprise, or be connectable to, a control system (e.g. a computer or processor) configured to perform the methods described herein (e.g. the methods set out in relation to FIGS. 3 and 4). For example, the sensor 100 may comprise, or be connectable to, a computer or processor-readable data carrier storing a computer program comprising instructions which, when the program is executed by a computer or processor, cause the computer or processor to carry out the methods described herein (e.g. the methods set out in relation to FIGS. 3 and 4).

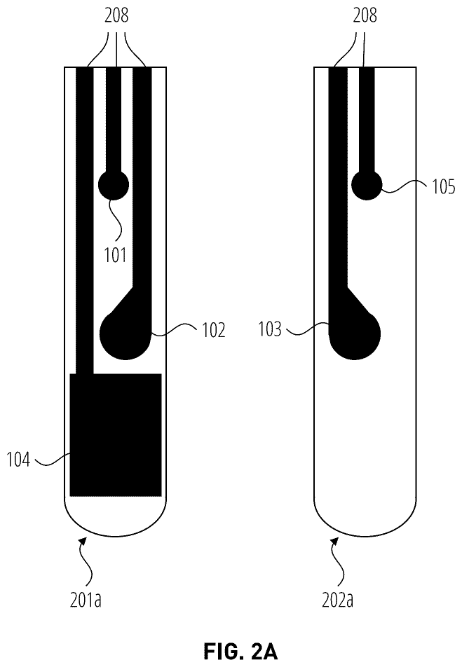

FIG. 2A illustrates an example electrode arrangement in an electrode area of the sensor 100. It will be appreciated that the sensor 100 illustrated in FIG. 2A is merely an example, and that the sensor and electrodes are not limited to the particular positions, shapes, sizes, or other characteristics shown in FIG. 2A. In certain examples, at least one of the electrodes may be disposed on a different side or surface of the sensor 100 to the other electrodes. For example, as illustrated in FIG. 2A, the first working electrode 101, the second working electrode 102, and the counter electrode 104 may be disposed on a first surface 201a (e.g. a front or top surface) facing a first direction, and the third working electrode 103 and the reference electrode 105 may be disposed on a second surface 202a (e.g. a back or bottom surface) facing a second direction. Arranging the electrodes on multiple surfaces may reduce the width of the sensor 100.

The arrangement of electrodes on different surfaces is not limited to the arrangement shown in FIG. 2A. For example, the electrodes can be arranged such that the second working electrode 102, the third working electrode 103, and the counter electrode 104 are disposed on the first surface 201a and such that the first working electrode 101 and the reference electrode 105 are disposed on the second surface 202a. Such an arrangement may improve the sensitivity of the sensor 100 by reducing interference between signals. For example, by arranging the first working electrode 101 on a different surface to the second working electrode 102, third working electrode 103, and counter electrode 104, interference between the potentiometric and amperometric signals may be reduced.

The electrodes may be electrically connected to connection lines 208 configured to connect the electrodes to a control system. For example, the connection lines 208 may directly electrically connect the electrodes to the control system, or may electrically connect to terminals (not illustrated) that are connectable to the control system.

In certain examples, each of the second working electrode 102 and the third working electrode 103 may have an area of a size that is equal to or larger than the size of the area of the first working electrode 101. In certain examples, each of the second working electrode 102 and the third working electrode 103 may have an area of a size that is between the size of the area of the first working electrode 101 and an area 1.5 times the size of the working electrode. In certain examples, each of the second working electrode 102 and the third working electrode 103 may have an area of a size that is 1.24 times the size of the first working electrode 101. For example, the first working electrode 101 may be a circle of diameter 0.42 mm (corresponding to an area of 0.14 mm²), and each of the second working electrode 102 and the third working electrode 103 may be a circle of diameter 0.47 mm (corresponding to an area of 0.17 mm²).

In certain examples, the reference electrode 105 may have an area of a size that is within 10% of the size of the first working electrode 101. In certain examples the reference electrode 105 may have an area of a size that is equal to the size of the area of the first working electrode 101. For example, the first working electrode 101 may be a circle of diameter 0.42 mm (corresponding to an area of 0.14 mm²), and the reference electrode 105 may be a circle of diameter 0.42 mm (corresponding to an area of 0.17 mm²).

In certain examples, the counter electrode 104 may have an area of a size that is larger than the size of the combined area of the second working electrode 102 and the third working electrode 103. In certain examples the counter electrode 104 may have an area of a size that is at least 1.2 times the size of the combined area of the second working electrode 102 and the third working electrode 103. For example, each of the second working electrode 102 and the third working electrode 103 may be a circle of diameter 0.47 mm (corresponding to an combined area of 0.35 mm²) and the counter electrode 104 may be a rectangle of length 0.72 mm and width 0.6 mm (corresponding to an area of 0.43 mm²).

The sensor 100 may be a needle-type sensor, wherein the sensor 100 comprises an electrode area, which is a portion of the sensor 100 on which the electrodes are arranged. The electrode area may have a thin width to facilitate implantation of the sensor 100 into tissue. As mentioned above, the width of the electrode area may be minimised by arranging electrodes on multiple surfaces. This allows a smaller electrode area while maximising the size of the electrodes, which may be important for improved sensitivity, for example.

In certain examples, the width of the sensor 100 (or the width of the portion of the sensor 100 on which the electrodes are arranged) may be at least 1.5 times the diameter of the first working electrode 101 and at most 2 times the diameter of the first working electrode 101. In certain examples the width of the sensor 100 (or the width of the portion of the sensor 100 on which the electrodes are arranged) may be at least 1.65 times the diameter of the first working electrode 101 and at most 1.75 times the diameter of the first working electrode 101. In certain examples, the width of the sensor 100 (or the width of the portion of the sensor 100 on which the electrodes are arranged) may be at least 1.66 times the diameter of the first working electrode 101 and at most 1.67 times the diameter of the first working electrode 101. For example, the first working electrode 101 may be a circle of diameter 0.42 mm and the width of the sensor 100 (or the width of the portion of the sensor 100 on which the electrodes are arranged) may be 0.7 mm.

In certain examples, the width of the sensor 100 may be 0.67 mm (within a certain manufacturing tolerance, for example ±20 μm).

FIG. 2B illustrates an example electrode arrangement of the sensor 100. In addition to first surface 201b and second surface 202b, FIG. 2B also includes a side view 203b of the electrode area of the sensor 100. It will be appreciated that the sensor 100 illustrated in FIG. 2B is merely an example, and that the sensor and electrodes are not limited to the particular positions, shapes, sizes, or other characteristics shown in FIG. 2B. In certain examples, at least one of the electrodes may be disposed on a different side or surface of the sensor 100 to the other electrodes. For example, as illustrated in FIG. 2B, the second working electrode 102, the third working electrode 103, and the counter electrode 104 may be disposed on a first surface 201a (e.g. a front or top surface) facing a first direction, and the first working electrode 101 and the reference electrode 105 may be disposed on a second surface 202b (e.g. a back or bottom surface) facing a second direction. Arranging the electrodes on multiple surfaces may reduce the width of the sensor 100.

Furthermore, arranging the electrodes on multiple surfaces may improve the sensitivity of the sensor 100 by reducing interference between signals. For example, by arranging the first working electrode 101 on a different surface to the second working electrode 102, third working electrode 103, and counter electrode 104, interference between the potentiometric and amperometric signals may be reduced.

The electrodes may comprise or be electrically connected to connection lines configured to connect the electrodes to a control system. For example, the connection lines may directly electrically connect the electrodes to the control system, or may electrically connect to terminals 210 that are connectable to the control system.

In certain examples, the electrodes may extend across the full width of the first surface 201b or second surface 202b. In this configuration, the electrodes may be referred to as lines of a certain height, with a width equal to the width of the sensor 100 (or the width of the portion of the sensor 100 on which the electrodes are arranged).

In certain examples, each of the second working electrode 102 and the third working electrode 103 may have an area of a size that is equal to or larger than the size of the area of the first working electrode 101. In certain examples, each of the second working electrode 102 and the third working electrode 103 may have an area of a size that is between the size of the area of the first working electrode 101 and an area 1.5 times the size of the working electrode. In certain examples, each of the second working electrode 102 and the third working electrode may have an area of a size that is 1.24 times the size of the first working electrode 101.

For example, for a sensor 100 with width of 0.7 mm (or in certain examples 0.67 mm) the first working electrode 101 may be a line of with a height of 0.5 to 1.5 mm (corresponding to an area of 0.34 to 1 mm²) and each of the second working electrode 102 and the third working electrode 103 may be of similar size.

In certain examples, the reference electrode 105 may have an area of a size that is within 10% of the size of the first working electrode 101. In certain examples the reference electrode 105 may have an area of a size that is equal to the size of the area of the first working electrode 101. For example, the first working electrode 101 may have a height of 0.5 to 1 mm, and the reference electrode 105 may be a line with a height of 0.5 to 1 mm (corresponding to an area of 0.34 to 0.67 mm²).

In certain examples, the counter electrode 104 may have an area of a size that is larger than the size of the combined area of the second working electrode 102 and the third working electrode 103. In certain examples the counter electrode 104 may have an area of a size that is at least 1.2 times the size of the combined area of the second working electrode 102 and the third working electrode 103. For example, each of the second working electrode 102 and the third working electrode 103 may be a line with a width of 0.5 to 1 mm (corresponding to an combined area of 0.68 to 1.5 mm²) and the counter electrode 104 may be a line with a height of 1.5 to 3 mm (corresponding to an area of 1.0 to 2.25 mm²).

The sensor 100 may be a needle-type sensor, wherein the sensor 100 comprises an electrode area, which is a portion of the sensor 100 on which the electrodes are arranged. By using the full width of the sensor, the electrode size, and therefore the signal, may be maximized, which may be important for improved sensitivity, for example.

Further, the manufacture of the sensors and application of the electrode functionalization formulations by line may be made possible by this configuration. Multiple sensors may be manufactured (e.g. in a roll-to-roll process) on a single sheet of substrate material by applying lines of material (e.g. conductive material and functionalization material for the electrodes, and insulating material to separate the electrodes if necessary) across the substrate in a first direction (e.g. horizontally) and then cutting the sheet in a second direction perpendicular to the first direction (e.g. vertically) to form a plurality of sensors. Compared to, e.g. dot dispensing of functionalization formulations, such a manufacturing technique may ensure a constant mass transfer of the functionalization formulations and may improve the manufacturing speed, reproducibility, and scalability of the sensors.

The electrodes may be layered on the sensor surface, as shown in the side view 203b. For example, considering the first surface 201b, a first conductive layer 211 may be disposed on the sensor surface (e.g. on the surface of a substrate) and may be partially covered with a first insulating layer 212 leaving an exposed end area to form an electrode (e.g. the second working electrode 102). A second conductive layer 213 may be disposed on the first insulating layer 212 and may be partially covered with a second insulating layer 214 leaving an exposed end area to form an electrode (e.g. the third working electrode 103). A third conductive layer 215 may be disposed on the second insulating layer 214 and may be partially covered with a third insulating layer 216 leaving an exposed end area to form an electrode (e.g. the counter electrode 104).

For example, considering the second surface 202b, a fourth conductive layer 217 may be disposed on the sensor surface and may be partially covered with a fourth insulating layer 218 leaving an exposed end area to form an electrode (e.g. the first working electrode 101). A fifth conductive layer 219 may be disposed on the fourth insulating layer 218 and may be partially covered with a fifth insulating layer 220 leaving an exposed end area to form an electrode (e.g. on which the reference electrode 105 is formed, for example with a layer of Ag/Cl covered in a conductive material, as described in more detail below).

Terminals 210 for connecting the electrodes to a control system may be formed at the other end of the sensor using a similar stepped arrangement. In this case, the connection lines connecting the electrodes to the terminals 210 are the conductive layers underneath the insulation layers. For example, the first insulating layer 212 may leave an exposed end area of the first conductive layer 211 at the other end of the sensor (i.e. the opposite end to the end with the electrode) to form a terminal 210 (e.g. terminal for the second working electrode 102). Second insulating layer 214 may leave an exposed end area of the second conductive layer 213 at the other end of the sensor (i.e. the opposite end to the end with the electrode) to form a terminal 210 (e.g. terminal for the third working electrode 103). Third insulating layer 216 may leave an exposed end area of the third conductive layer 215 at the other end of the sensor (i.e. the opposite end to the end with the electrode) to form a terminal 210 (e.g. terminal for the counter electrode 104). Fourth insulating layer 218 may leave an exposed end area of the fourth conductive layer 217 at the other end of the sensor (i.e. the opposite end to the end with the electrode) to form a terminal 210 (e.g. terminal for the first working electrode 101). Fifth insulating layer 220 may leave an exposed end area of the fifth conductive layer 219 at the other end of the sensor (i.e. the opposite end to the end with the electrode) to form a terminal 210 (e.g. terminal for the reference electrode 105).

However, it will be appreciated that the arrangement and dimensions of the sensor 100 and electrodes shown in FIGS. 2A and 2B and described above are merely examples, and that any suitable sensor and electrode arrangement and dimensions may be used. For example, although FIG. 2A illustrates the first working electrode 101 on the first surface 201a and the third working electrode 103 on the second surface 202a, the electrodes may alternatively be arranged with the third working electrode 103 on the first surface 201a and the first working electrode 101 on the second surface 202a, similar to FIG. 2B.

In certain examples, a pre-measurement period during which the second working electrode 102 and/or third working electrode 103 are polarized may occur during at least part of the potentiometric measurement at the first working electrode 101. In order to perform an amperometric measurement, a voltage must be applied to polarize the working electrode. For example, the voltage may be the same voltage that will be used to perform the amperometric measurement. However, for an initial period of the voltage application, the measured current will not correspond to the analyte concentration. That is, a pre-measurement period is required before measurement of the analyte concentration can begin. If the voltage application at the second working electrode 102 and/or third working electrode 103 is stopped, for example to perform a potentiometric measurement using the first working electrode 101, it becomes necessary to use a further pre-measurement period to re-polarize the second working electrode 102 and/or third working electrode 103 before measurement can begin again. Thus while it may be possible to share the same reference electrode 105 between potentiometric and amperometric techniques by first performing the potentiometric measurement at the first working electrode 101 then switching to performing the amperometric measurement at the second working electrode 102 and/or third working electrode 103, the need for a pre-measurement period to polarize the second working electrode 102 and/or third working electrode 103 is inconvenient and may limit the frequency of measurement of the sensor 100. To increase the measurement frequency, the pre-measurement polarization at the second working electrode 102 and/or third working electrode 103 may therefore take place during the potentiometric measurement at the first working electrode 101. Similarly, the pre-measurement polarization at the third working electrode 103 may take place during the amperometric measurement at the second working electrode 102. However, in other examples, the pre-measurement polarization at the second working electrode 102 and/or third working electrode 103 may take place before the potentiometric measurement at the first working electrode 101, for example so that measurement of the first signal, second signal, and third signal may begin simultaneously.

Furthermore, if the amperometric measurement is linked to an enzyme cascade in which the analyte is converted to a product able to be oxidized or reduced on the second working electrode 102 and/or third working electrode 103 (as described in more detail below), the product may accumulate in the vicinity of the second working electrode 102 and/or third working electrode 103 when the second working electrode 102 and/or third working electrode 103 is not polarized (i.e. when the voltage is not being applied), and the sensor 100 may become saturated. Therefore, in order to use the sensor 100 for continuous monitoring of the second analyte concentration and/or the third analyte concentration, continuous polarization of the second working electrode 102 and/or third working electrode 103 is advantageous. That is, stopping the voltage application at the second working electrode 102 and/or third working electrode 103 (for example to switch to potentiometric measurement at the first working electrode 101) may be detrimental to the ability of the sensor 100 to be used for continuous monitoring of the second analyte concentration and/or third analyte concentration.

The pre-measurement polarization voltage may be applied using the same reference electrode 105 as is being used to perform the potentiometric measurement with the first working electrode 101. When the pre-measurement polarization occurs at both the second working electrode 102 and the third working electrode 103 simultaneously, the same counter electrode 104 may be used for the polarization.

In certain examples, the first analyte may be an ion, for example potassium, sodium, magnesium, urea, or calcium. In some examples, the first analyte may be hydrogen ions, such that the first working electrode 101 may function as a pH sensor. However, it will be appreciated that these are just examples, and the first analyte may be any substance that can be detected using potentiometry, for example urea, Na, Mg2, CO2, Cl, etc.

In certain examples, the first working electrode 101 may be functionalized to detect a potential indicative of the first analyte concentration. That is, the first working electrode 101 may be functionalized such that the potential measured at the first working electrode 101 (relative to the reference electrode 105) is indicative of the concentration of the first analyte in a sample to be measured.

In the case that the first analyte is an ion, the first working electrode 101 may be functionalized with an ion selective membrane corresponding to the first analyte. For example, the first working electrode 101 may be a solid contact ion selective electrode corresponding to the first analyte.

The ion selective membrane may comprise a polymer matrix, an ionophore, a plasticiser, and an ion exchanger. For example, in the case that the first analyte is potassium ions, the ionophore may comprise valinomycin, the polymer matrix may comprise polyvinyl chloride, the plasticizer may comprise bis(2-ethylhexyl) adipate (DOA), and the ion exchanger may comprise potassium tetrakis-(4-chlorophenyl)-borate (KTpClPB) or potassium tetrakis-[3,5-bis(trifluoromethyl)phenyl]-borate (KTFPB).

In certain examples, the second analyte may be a metabolite.

In one example, the second analyte may be creatinine, creatine, NT-proBNP, BNP, glucose, lactate, ketone, alcohol, or oxygen.

In certain examples, the third analyte may be a metabolite.

In one example, the third analyte may be creatinine, creatine, NT-proBNP, BNP, glucose, lactate, ketone, alcohol, or oxygen.

In certain examples, the second working electrode 102 may be functionalized to detect a current indicative of the second analyte concentration. That is, the second working electrode 102 may be functionalized such that the current measured at the second working electrode 102 is indicative of the concentration of the second analyte in a sample to be measured.

In certain examples, the third working electrode 103 may be functionalized to detect a current indicative of the third analyte concentration. That is, the third working electrode 103 may be functionalized such that the current measured at the third working electrode 103 is indicative of the concentration of the third analyte in a sample to be measured.

The measured current at the second working electrode 102 may result from the oxidation or reduction of the second analyte at the second working electrode 102. In order to increase the selectivity, in certain examples the second working electrode 102 may be functionalized with biological components that allow selective recognition or conversion of an analyte, such as enzymes, antibodies or whole cells. For example, the second working electrode 102 may have enzymes immobilized on the electrode surface where the main function of the enzyme is to catalyze the conversion of an electrochemically inactive substrate (e.g. the second analyte) into an electroactive species that can be monitored amperometrically.

Immobilization of the enzymes on the second working electrode surface may be done by different mechanisms, depending on the molecular structure of the enzyme and the electrode material. Covalent-binding based immobilization may provide more stable electrodes due to the firm binding of the receptor molecules. Non-covalent immobilization methods are also possible, and physical adsorption may be a simple way to fix bioactive substances to the electrode surface and may suitable for the production of low-cost sensors for disposable applications. The second working electrode 102 may be further stabilized by enclosing the enzyme layer through an analyte-permeable polymer membrane as a thin film. The incorporation of these analyte-permeable polymer membranes on the reaction layer may also act as a protective barrier and increase the selectivity of the sensor 100. The polymer membrane may prevent large molecules in biological samples from entering the reaction layer and causing interference.

In certain examples, if the second analyte is glucose, the second working electrode 102 may be functionalized with the enzyme glucose oxidase. The glucose may be detected indirectly via the conversion of glucose to hydrogen peroxide by the glucose oxidase. The catalyzed hydrogen peroxide is then monitored by an electrochemical anodic reaction. The current flowing during this anodic reaction is proportional to the glucose concentration. With the help of a calibration of the glucose sensor, a quantitative determination of an unknown glucose concentration is possible.

In certain examples, if the second analyte is creatinine, the second working electrode 102 may be functionalized with the three enzymes creatininase, creatinase, and sarcosine oxidase, which form a three enzyme cascade. The three-enzyme cascade releases glycine and hydrogen peroxide in a three-step conversion of creatinine according to the reactions set out in Equations 1-3 below:

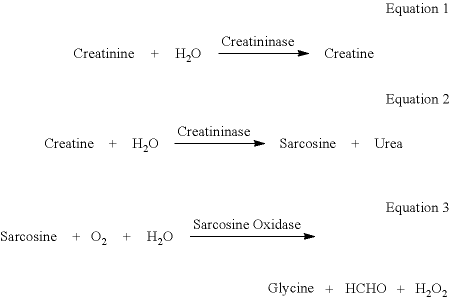 [SMILES: O ; O ; "O2" ; O ; "HCHO" ; "H2O2"]

In the last reaction step (see Equation 3), the consumption of electrochemically detectable oxygen and the release of hydrogen peroxide (H₂O₂) takes place. Therefore, the creatinine concentration can be determined using two detection methods. The decrease in oxygen, which is proportional to the creatinine concentration, can be detected with an oxygen electrode. The second method involves the detection of H₂O₂ release. The catalyzed hydrogen peroxide is then electrochemically oxidized to oxygen at the second working electrode 102, causing a transfer of two electrons that can be measured as an electric current at a constant applied potential (see Equation 4). The current measured is proportional to the concentration of the second analyte.

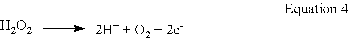

Similar to the second working electrode 102, the measured current at the third working electrode 103 may result from the oxidation or reduction of the third analyte at the third working electrode 103. In order to increase the selectivity, in certain examples the third working electrode 103 may be functionalized with biological components that allow selective recognition or conversion of an analyte as described above for the second working electrode 102.

In certain examples, if the third analyte is creatine, the third working electrode 103 may be functionalized with the two enzymes creatinase, and sarcosine oxidase, which form a two enzyme cascade. The two-enzyme cascade releases glycine and hydrogen peroxide in a two-step conversion of creatine according to the reactions set out in Equations 2-3 above.

In the last reaction step (see Equation 3), the consumption of electrochemically detectable oxygen and the release of hydrogen peroxide (H₂O₂) takes place, and the creatine concentration can be determined as described above for creatinine.

In certain examples, at least two of the first analyte, second analyte, and third analyte may be different biomarkers relevant to a particular health condition. For example, if the particular health condition is kidney failure, the first analyte may be potassium and the second analyte may be creatinine. In certain examples, when the first analyte and second analyte are relevant to a particular health condition, the third analyte may also be relevant to the same health condition. For example, if the particular health condition is diabetes and its comorbidities the first analyte may be hydrogen ions (for determining pH) or another relevant ion, the second analyte may be glucose, and the third analyte may be ketones or lactate.

In certain examples, the analytes may be different biomarkers related to linked health conditions. In certain examples, the analytes may be different biomarkers related to effects of treatments (for example, at least one analyte may be related to monitoring the intended effect of the treatment, or to monitoring the condition that the treatment is intended to treat, and at least one analyte may be related to monitoring side effects of the treatment). For example, patients with heart failure may receive treatments (such as RAAS-i therapy) which may have side effects such as worsening renal function and electrolyte disturbances. In such an example, the second analyte may be NT-proBNP for monitoring heart failure (i.e. monitoring the intended effect of the treatment on the condition), while the first and third analytes may be potassium and creatinine, respectively, for monitoring kidney function (i.e. monitoring for adverse side effects of the treatment). By simultaneously monitoring the intended effect and the side effects of treatment, the treatment may be used more effectively. For example, larger/more frequent doses of the treatment may be used for a patient based on the monitoring showing less indication of harmful side effects.

In certain examples, the third signal may be used to correct the second signal. For example, the third analyte concentration may be used to calculate or refine/correct the calculation of the second analyte concentration.

For example, if the second analyte is creatinine, the third analyte may be creatine. In vivo creatinine measurement could be affected by interference with endogenous creatine. In the enzyme reaction of creatinine determination, creatine is produced as an intermediate, which is converted to sarcosine by creatinase, as described above (see Equations 1-3). In this intermediate stage, there will be interference from endogenous creatine. Consequently, a more accurate measurement of the creatinine concentration can be performed by simultaneously measuring the second signal with the second working electrode 102 and the third signal with the third working electrode 103 and subtracting the third signal from the second signal to remove the interference from the creatine.

In certain examples, the first analyte is potassium, the second analyte is creatinine, and the third analyte is creatine. As described above, the third signal (indicative of creatine concentration) may be used to obtain a more accurate calculation of the creatinine concentration. Simultaneous measurement of potassium concentration and creatinine concentration may be used for health monitoring of patients with kidney failure, for example.

In certain examples, the counter electrode 104 may have an area that is larger than the combined area of the working electrodes that may be used simultaneously. For example, when the counter electrode 104 is to be used to detect the second signal and third signal simultaneously, the counter electrode 104 may have an area larger than the combined area of the second working electrode 102 and the third working electrode 103. In certain examples, a larger area may comprise an area at least 1.5 times larger than the combined area of the working electrodes that may be used simultaneously. By using a counter electrode 104 with a larger area, the ability of the sensor 100 to apply constant potentials at the second working electrode 102 and third working electrode 103 during simultaneous amperometric measurement may be improved.

FIG. 3 illustrates an example method of detecting a plurality of analytes in bodily fluid using a sensor 100 comprising a first working electrode 101, a second working electrode 102, a third working electrode 103, a counter electrode 104, and a reference electrode 105. The method may be performed using any of the example sensors 100 described above, and it will be appreciated that all of the features described above are compatible with the method.

At step 301, the method comprises detecting a first signal indicative of a first analyte concentration using potentiometry at the first working electrode 101. That is, a first signal (e.g. a potential) may be detected using the first working electrode 101 wherein the first signal may be used to calculate the concentration of the first analyte in a sample solution which the first working electrode 101 contacts.

At step 302, the method comprises detecting a second signal indicative of a second analyte concentration using amperometry at the second working electrode 102. That is, a second signal (e.g. a current) may be detected using the second working electrode 102 wherein the second signal may be used to calculate the concentration of the second analyte in a sample solution which the second working electrode 102 contacts.

At step 303, the method comprises detecting a third signal indicative of a third analyte concentration using amperometry at the third working electrode 103. That is, a third signal (e.g. a current) may be detected using the third working electrode 103 wherein the third signal may be used to calculate the concentration of the third analyte in a sample solution which the third working electrode 103 contacts.

The first analyte, second analyte, and third analyte are different analytes.

The first signal, second signal, and third signal are detected using the same reference electrode 105.

At least part of the detection of the second signal (i.e. step 302) occurs simultaneously with the detection of the first signal (i.e. step 301). That is, the first signal may be detected at the same time as the second signal.

The second signal and third signal are detected using the same counter electrode 104.

At least part of the detection of the second signal (i.e. step 302) occurs simultaneously with the detection of the third signal (i.e. step 303). That is, the second signal may be detected at the same time as the third signal.

In certain examples, at least part of the detection of the third signal (i.e. step 303) may occur simultaneously with detection of the first signal and the second signal (i.e. steps 301 and 302). That is, the first signal, second signal, and third signal may be detected at the same time.

FIG. 4 illustrates an example method of detecting a plurality of analytes in bodily fluid using a sensor 100 comprising a first working electrode 101, a second working electrode 102, a third working electrode 103, a counter electrode 104, and a reference electrode 105. The method may be performed using any of the example sensors 100 described above, and it will be appreciated that all of the features described above are compatible with the method.

At step 401, the method comprises detecting a first signal indicative of a first analyte concentration using potentiometry at the first working electrode 101. Step 401 is the same as step 301 of FIG. 3, and the further description of step 301 applies also to step 401.

At step 402, the method comprises detecting a second signal indicative of a second analyte concentration using amperometry at the second working electrode 102. Step 402 is the same as step 302 of FIG. 3, and the further description of step 302 applies also to step 402.

At step 403, the method comprises detecting a third signal indicative of a third analyte concentration using amperometry at the third working electrode 103. Step 403 is the same as step 303 of FIG. 3, and the further description of step 303 applies also to step 403.

At step 404, the method further comprises monitoring the first analyte concentration, based on the first signal.

At step 405, the method further comprises monitoring the second analyte concentration, based on the second signal.

The first signal, second signal, and third signal are detected using the same reference electrode 105.

At least part of the detection of the second signal (i.e. step 402) occurs simultaneously with the detection of the first signal (i.e. step 401). That is, the first signal may be detected at the same time as the second signal.

The second signal and third signal are detected using the same counter electrode 104.

At least part of the detection of the second signal (i.e. step 402) occurs simultaneously with the detection of the third signal (i.e. step 403). That is, the second signal may be detected at the same time as the third signal.

In certain examples, the method may further comprise monitoring the third analyte concentration, based on the third signal.

In certain examples, step 405 may comprise monitoring the second analyte concentration based the third signal (in addition to the second signal) by correcting the second signal using the third signal. That is, the third signal may be used to correct the second signal to more accurately monitor the second analyte concentration.

In certain examples, the first working electrode 101, second working electrode 102 and/or third working electrode 103 of the sensor 100 may comprise gold electrodes. In certain examples, the first working electrode 101, second working electrode 102 and/or third working electrode 105 may comprise carbon electrodes. In certain examples, the reference electrode 105 may comprise an Ag/AgCl reference electrode. Optionally, the reference electrode 105 may be covered with a layer of carbon to prevent leaching. For example, the Ag/AgCl reference electrode 105 may be covered with a layer of carbon to prevent silver leaching. In certain examples, the counter electrode 104 may be a carbon electrode. However, it will be appreciated that these electrode materials are merely examples, and that any suitable mixture of electrode materials may be used for the various electrodes of the sensor 100.

In certain examples, a catalyst may be added to at least one of the working electrode materials. For example, if the second working electrode 102 is functionalized to detect a signal indicative of creatinine concentration, manganese dioxide may be included in the second working electrode 102 material. Advantageously, manganese dioxide leads to catalysis of the reaction in Equation 4 and thus lowers the necessary oxidation potential for the detection of H₂O₂.

In certain examples, the membrane solution for functionalizing the first working electrode 101 to detect a potential indicative of potassium concentration may be prepared according to Table 1.

TABLE 1

| | Percentage range by weight | Example weight |
|---|---|---|
| Substance | (%) | (mg) |
| Valinomycin | 0.4-0.6 | 17.0 |
| Potassium tetrakis (4- | 0.1-0.2 | 5.0 |
| chlorophenyl) borate | | |
| Poly(vinyl chloride) | 8-12 | 313.0 |
| THF | 70-85 | 1250.0 |
| Bis(2-ethylhexyl) adipate | 8-12 | 350.0 |

In certain examples, a creatinine electrode formulation according to Table 2 may be used for functionalizing the second working electrode 102 to detect a current indicative of creatinine concentration.

TABLE 2

| | | |
|---|---|---|
| | Percentage range by | Example weight |
| Substance | weight (%) | (mg) |

Glycerol-Tween solution (solvent solution for the enzymes)

| | | |
|---|---|---|
| Glycerol | 8-12 | 600.0 |
| Tween 20 | 0.3-0.5 | 21.6 |
| Ultra-pure water | 85-95 | 5378.4 |

Enzyme solution

| | | |
|---|---|---|
| Creatininase | 8-12 | 200.0 |
| Creatinase | 20-30 | 500.0 |
| Sarcosine oxidase | 5-10 | 125.0 |
| Glycerol-Tween solution | 55-65 | 1175.0 |

In addition to the creatinine enzyme working solution, a cross-linking working solution of glutaraldehyde may be prepared for the second working electrode 102 according to Table 3.

TABLE 3

| | Percentage range by weight | Example weight |
|---|---|---|
| Substance | (%) | (mg) |
| 25 wt % Glutaraldehyde | 1-2 | 71.2 |
| solution | | |
| Ultra-pure water | 98-99 | 4928.8 |

In certain examples, a creatine electrode formulation according to Table 4 may be used for functionalizing the third working electrode 103 to detect a current indicative of creatine concentration.

TABLE 4

| | | |
|---|---|---|
| | Percentage range by | Example weight |
| Substance | weight (%) | (mg) |

Glycerol-Tween solution (solvent solution for the enzymes)

| | | |
|---|---|---|
| Glycerol | 8-12 | 600.0 |
| Tween 20 | 0.3-0.5 | 21.6 |
| Ultra-pure water | 85-95 | 5378.4 |

Enzyme solution

| | | |
|---|---|---|
| Creatinase | 20-30 | 500.0 |
| Sarcosine oxidase | 5-10 | 125.0 |
| Glycerol-Tween solution | 55-65 | 1175.0 |

In addition to the creatine enzyme working solution, a cross-linking working solution of glutaraldehyde may be prepared for the third working electrode 103 according to Table 3.

Polyvinylpyrrolidone (PVP) based polymer membrane may be used for the preparation of the first working electrode 101, second working electrode 102, and/or third working electrode 103 and may be prepared according to Table 5.

TABLE 5

| | Target PVP | Example | |
|---|---|---|---|
| | concentration range | concentration | |
| Substance | (mg/ml) | (mg/(EtOH)mL | Example amount |

PVP

| | | | | |
|---|---|---|---|---|
| PVP | 80-120 | 153.52 | 3.40 | g |
| PEGDGE500 | 15-30 | 23.90 | 1.85 | mL |

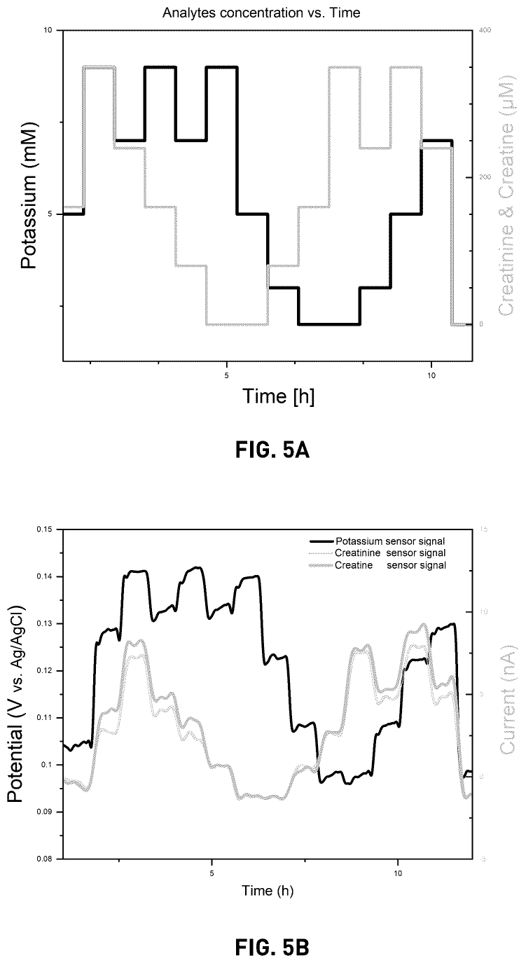

FIGS. 5A and 5B show experimental results demonstrating use of the sensor 100 to simultaneously measure potassium at the first working electrode 101 using potentiometry, creatinine at the second working electrode 102 using amperometry, and creatine at the third working electrode 103 using potentiometry, wherein the measurements shared a single counter electrode 104 and single reference electrode 105.

FIG. 5A shows how the concentration of analytes in the sample solution was varied over time. FIG. 5B shows the measured potential at the first working electrode 101 (potassium sensor signal), measured current at the second working electrode 102 (creatinine sensor signal), and the measured current at the third working electrode 103 (creatine sensor signal), for the changes in concentration shown in FIG. 5A.

1. A sensor for detecting a plurality of analytes in bodily fluid, the sensor comprising electrodes including a first working electrode, a second working electrode, a third working electrode, a counter electrode, and a reference electrode;
wherein the first working electrode is configured to detect a first signal indicative of a first analyte concentration using potentiometry;
wherein the second working electrode is configured to detect a second signal indicative of a second analyte concentration using amperometry;
wherein the third working electrode is configured to detect a third signal indicative of a third analyte concentration using amperometry;
wherein the first analyte, the second analyte, and third analyte are different analytes;
wherein the first signal, the second signal, and the third signal are detected using the same reference electrode;
wherein at least part of the detection of the first signal occurs simultaneously with the detection of the second signal;
wherein the second signal and third signal are detected using the same counter electrode;
wherein at least part of the detection of the second signal occurs simultaneously with the detection of the third signal; and
wherein at least one of the second working electrode and third working electrode is continuously polarized by application of a polarization voltage during detection of the first signal, second signal and third signal.

2. The sensor of claim 1, wherein at least part of the detection of the third signal occurs simultaneously with detection of the first signal and the second signal.

3. The sensor of claim 1, wherein the first analyte is an ion.

4. The sensor of claim 3 wherein the ion is one of potassium, sodium, magnesium, urea, or calcium.

5. The sensor of claim 1, wherein the second analyte is a metabolite.

6. The sensor of claim 5, wherein the metabolite is one of creatinine, creatine, NT-proBNP, BNP, glucose, lactate, ketone, alcohol, or oxygen.

7. The sensor of claim 1, wherein the third analyte is a metabolite.

8. The sensor of claim 7, wherein the third analyte is one of creatinine, creatine, NT-proBNP, BNP, glucose, lactate, ketone, alcohol, or oxygen.

9. The sensor of claim 1, wherein the third signal is used to correct the second signal.

10. A method of detecting a plurality of analytes in bodily fluid using a sensor comprising a first working electrode, a second working electrode, a third working electrode, a counter electrode, and a reference electrode, the method comprising:
detecting a first signal indicative of a first analyte concentration using potentiometry at the first working electrode;
detecting a second signal indicative of a second analyte concentration using amperometry at the second working electrode; and
detecting a third signal indicative of a third analyte concentration using amperometry at the third working electrode;
wherein the first analyte, the second analyte, and the third analyte are different analytes;
wherein the first signal, the second signal, and the third signal are detected using the same reference electrode;
wherein at least part of the detection of the first signal occurs simultaneously with the detection of the second signal;
wherein the second signal and third signal are detected using the same counter electrode;
wherein at least part of the detection of the second signal occurs simultaneously with the detection of the third signal; and
wherein the method further comprises continuously polarizing at least one of the second working electrode and third working electrode by application of a polarization voltage during detection of the first signal, second signal and third signal.

11. The method of claim 10, wherein at least part of the detection of the third signal occurs simultaneously with detection of the first signal and the second signal.

12. The method of claim 10, further comprising:
monitoring the first analyte concentration, based on the first signal;
monitoring the second analyte concentration, based on the second signal; and
monitoring the third analyte concentration, based on the third signal.

13. The method of claim 10, further comprising:
monitoring the first analyte concentration, based on the first signal;
monitoring the second analyte concentration, based on the second signal and the third signal, by correcting the second signal using the third signal.

14. The method of claim 10, wherein at least one of the first, second, or third analyte concentrations is indicative of a health condition, and wherein at least one other of the first, second, or third analyte concentrations is indicative of a side effect of medication for treating the health condition.

15. The method of claim 13, wherein the first analyte is potassium, the second analyte is creatinine, and the third analyte is creatine.

16. The method of claim 10, wherein the sensor is a needle-type sensor comprising an electrode area on which the first working electrode, second working electrode, third working electrode, counter electrode, and reference electrode are arranged;
wherein the electrode area comprises a first surface facing a first direction on which at least one of the first working electrode, second working electrode, third working electrode, counter electrode, and reference electrode are arranged;
wherein the electrode area comprises a second surface facing a second direction, opposite to the first direction, on which is arranged at least one of the first working electrode, second working electrode, third working electrode, counter electrode, and reference electrode that is not arranged on the first surface.

17. The sensor or method of claim 16, wherein the first working electrode is arranged on the second surface and the second working electrode, third working electrode, and counter electrode are arranged on the first surface.

18. The sensor or method of claim 16, wherein at least one of the electrodes arranged on the first surface extends across the full width of the first surface and/or at least one of the electrodes arranged on the second surface extends across the full width of the second surface.

19. The sensor or method of claim 18, wherein each of the electrodes arranged on the first surface extends across the full width of the first surface and each of the electrodes arranged on the second surface extends across the full width of the second surface.
